(19) United States
(12) Patent Application Publication
(10) Pub. No.: US 2010/0286194 A1
(43) Pub. Date: Nov. 11, 2010

(54) METHOD FOR TREATING ADDICTION

(75) Inventors: Naheed Mirza, West Lothian (GB); Jens Damsgaard, Lyngby (DK)

Correspondence Address: BIRCH STEWART KOLASCH & BIRCH, PO BOX 747, FALLS CHURCH, VA, 22040-0747, US

(73) Assignee: Neurosearch A/S, Ballerup (DK)

(21) Appl. No.: 12/743,512

(22) PCT Filed: Nov. 19, 2008
(86) PCT No.: PCT/EP08/65802

§ 371 Date: Jul. 22, 2010

Related U.S. Application Data
(60) Provisional application No. 60/989,322, filed on Nov. 20, 2007.

(30) Foreign Application Priority Data
Nov. 20, 2007 (DK) PA200701642

(51) Int. Cl. A61K 31/46; A61P 25/32; A61P 25/34; A61P 25/36; A61P 25/30

(52) U.S. Cl. 514/304

(57) ABSTRACT

A method for treating addiction, in particular nicotine addiction, alcohol addiction and drug addiction, such as opioid addiction. The invention furthermore relates to novel pharmaceutical compositions for the treatment of addiction comprising a therapeutically effective amount of a compound of formula I.

### TECHNICAL FIELD

This invention relates to a method for treating addiction, in particular nicotine addiction, alcohol addiction, drug addiction or other addiction. The invention furthermore relates to novel pharmaceutical compositions comprising a therapeutically effective amount of a compound of formula I.

### BACKGROUND ART

It is generally accepted that addiction is a disease, a state of physiological or psychological dependence or devotion to something manifesting as a condition in which medically significant symptoms liable to have a damaging effect are present. Thus smoking is one of the major causes of lung disease, heart disease, and certain forms of cancer, while excessive alcohol consumption may lead to liver failure or acute respiratory failure.

Physical dependence on a substance is defined by the appearance of characteristic withdrawal symptoms when the substance or behavior is suddenly discontinued. Alcohol and nicotine are well known for their ability to induce physical dependence. So, while physical dependency can be a major factor in the psychology of addiction and often is the primary reason for the continuation of an addiction, the initial primary attribute of an addictive substance is usually its ability to induce pleasure, although with continued use the goal is more to relieve the anxiety caused by the absence of a given addictive substance, causing it to become used compulsively. Further, the physical dependency of the nicotine addict on the substance itself becomes an overwhelming factor in the continuation of use.

The speed with which a given individual becomes addicted to various substances varies with the substance, the frequency of use, the means of ingestion, the intensity of pleasure or euphoria, and the individual's genetic and psychological susceptibility. Some alcoholics exhibit alcoholic tendencies from the moment of first intoxication, while most people can drink socially without ever becoming addicted.

WO 97/30997 discloses tropane derivatives, their preparation and use as monoamine neurotransmitter, i.e. dopamine, serotonin, and noradrenaline, reuptake inhibitors.

### SUMMARY OF THE INVENTION

It has surprisingly been shown that a compound of formula I below in a specified dosage range can be used for treating addiction.

In its first aspect the invention provides a method for treating addiction comprising administering to a human a composition comprising a compound of formula I, any of its stereoisomers or any mixture of its stereoisomers, or a pharmaceutically acceptable salt thereof in a therapeutically-effective amount in the range about 0.1-2 mg API daily.

In another aspect the invention relates to a pharmaceutical composition effective for treating addiction in a human, said composition comprising a therapeutically-effective amount in the range about 0.1-2 mg API daily of a compound of formula I, any of its stereoisomers or any mixture of its stereoisomers, or a pharmaceutically acceptable salt thereof in admixture with one or more pharmaceuticaly acceptable adjuvants, excipients, carriers and/or diluents.

Other objects of the invention will be apparent to the person skilled in the art from the following detailed description and examples.

### DETAILED DISCLOSURE OF THE INVENTION

In its first aspect the invention provides a method for treating addiction comprising administering to a human a composition comprising a compound of formula I

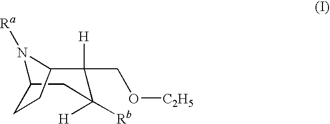

wherein
R^{a} represents hydrogen or alkyl;
R^{b} represents a dihalophenyl group;
any of its stereoisomers or any mixture of its stereoisomers, or a pharmaceutically acceptable salt thereof in a therapeutically-effective amount in the range 01-2 mg API daily.

The compounds of formula I for use according to the invention are described in WO 97/30997 (NeuroSearch A/S). The compounds may be prepared by conventional methods for chemical synthesis, e.g. those described in WO 97/30997.

In one embodiment of the compound of formula I, R^{a} represents hydrogen or methyl. In a special embodiment, R^{a} represents hydrogen. In a further embodiment, R^{a} represents methyl.

In a further embodiment of the compounds of formula I, R^{b} represents dichlorophenyl. In a special embodiment, R^{b} represents 3,4-dichlorophenyl.

In a still further embodiment, the compound of formula I is
tesofensine [(1R,2R,3S,5S)-3-(3,4-dichlorophenyl)-2-(ethoxymethyl)-8-methyl-8-azabicyclo[3.2.1]octane]; or
(1R,2R,3S,5S)-3-(3,4-dichlorophenyl)-2-(ethoxymethyl)-8-azabicyclo[3.2.1]octane;
   or a pharmaceutically acceptable salt thereof.

In a special embodiment, the compound of formula I is tesofensine or a pharmaceutically acceptable salt thereof. In a further special embodiment, the compound of formula I is the citrate salt of tesofensine.

### DEFINITION OF SUBSTITUENTS

In the context of this invention halo represents fluoro, chloro, bromo or dodo.

In the context of this invention an alkyl group means a straight chain or branched chain of one to six carbon atoms, including but not limited to, methyl, ethyl, propyl, isopropyl, butyl, isobutyl, t-butyl, pentyl, and hexyl; methyl, ethyl, propyl and isopropyl are preferred groups.

### Pharmaceutically Acceptable Salts

The active compounds for use according to the invention may be provided in any form suitable for the intended administration. Suitable forms include pharmaceutically (i.e. physiologically) acceptable salts, and pre- or prodrug forms of the compound of formula I for use according to the invention.

Examples of pharmaceutically acceptable addition salts include, without limitation, the non-toxic inorganic and organic acid addition salts such as the hydro-chloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a compound of formula I for use according to the invention include, without limitation, the sodium, the potassium, the calcium, the magnesium, the zinc, the aluminium, the lithium, the choline, the lysinium, and the ammonium salt, and the like, of a compound of formula I for use according to the invention containing an anionic group. Such cationic salts may be formed by procedures well known and described in the art.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

Examples of pre- or prodrug forms of the compound of formula I for use according to the invention include examples of suitable prodrugs of the compounds of formula I modified at one or more reactive or derivatizable groups of the parent compound. Of particular interest are compounds modified at a carboxyl group, a hydroxyl group, or an amino group. Examples of suitable derivatives are esters or amides.

The compound of formula I for use according to the invention may be provided in dissoluble or indissoluble forms together with a pharmaceutically acceptable solvent such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

### Steric Isomers

It will be appreciated by those skilled in the art that the compounds of formula I may exist in different stereoisomeric forms—including enantiomers, diastereomers and cis-trans-isomers.

The invention includes all such stereoisomers and any mixtures thereof including racemic mixtures.

### Dosage

The dosage of a compound of formula I is determined as the API (Active Pharmaceutical Ingredient), i.e. calculated as the'free base.

In the method according to the invention the compound of formula I, any of its stereoisomers or any mixture of its stereoisomers, or a pharmaceutically acceptable salt thereof is administered to a human in need thereof in a therapeutically-effective amount in the range of about 0.1-2 mg API daily.

The actual dosage of each of the active ingredients depends on the nature and severity of the disease being treated, the exact mode of administration, form of administration and is within the discretion of the physician, and may be varied by titration of the dosage to the particular circumstances of this invention to produce the desired therapeutic effect. However, a daily dosage in the range from about 0.1-2 mg API daily, preferably of from about 0.25-1 mg API daily, especially 0.25, 0.5 or 1.0 mg API daily, is suitable for therapeutic treatments.

### Pharmaceutical Compositions

While the compounds for use according to the invention may be administered in the form of the raw compound, it is preferred to introduce the active ingredients, optionally in the form of physiologically acceptable salts, in a pharmaceutical composition together with one or more adjuvants, excipients, carriers, buffers, diluents, and/or other customary pharmaceutical auxiliaries.

Thus in another aspect the present invention provides a pharmaceutical composition effective for treating addiction in a human, said composition comprising a therapeutically-effective amount in the range of about 0.1-2 mg API daily of a compound of formula I

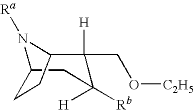

wherein
R^{a} represents hydrogen or alkyl;
R^{b} represents a dihalophenyl group;
any of its stereoisomers or any mixture of its stereoisomers, or a pharmaceutically acceptable salt thereof in admixture with one or more pharmaceuticaly acceptable adjuvants, excipients, carriers and/or diluents.

The one or more adjuvants, excipients, carriers, buffers, diluents, and/or other customary pharmaceutical auxiliaries must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragée, in powder, or in liquid form, topically such as by inhalation, by patch, enterally, such as by suppository, and parenteral administration, in particular cutaneous, subcutaneous, intramuscular, or intravenous injection. The pharmaceutical composition of the invention can be manufactured by the skilled person by use of standard methods and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

### Biological Activity

The compound of formula I may be used in the method according to the invention for treating addiction, in particular addiction selected from the group comprising nicotine addiction, alcohol addiction, drug addiction, such as opioid addiction, nicotine craving, alcohol craving, drug craving, such as opioid craving, food craving, craving for fatty food and craving for carbohydrate rich food. Furthermore, the compound of formula I may be used for the relief of nicotine, alcohol or drug withdrawal symptoms, for maintenance of abstinence from nicotine, alcohol or drugs, for detoxication or maintenance treatment of nicotine, alcohol or opioid addiction or for the blockade of the effects of exogenously administered opioids. Also, the compound of formula I may be used for decreasing the desire to consume alcohol, for decreasing the desire to use drugs, such as opioids, for decreasing food craving, for decreasing craving for fatty food or for decreasing craving for carbohydrate rich food. Still further, the compound of formula I may also be used for preventing relapse into addiction.

The activity of the compound of formula I in the method according to the invention will be illustrated by means of the following examples using the compound tesofensine ([(1R,2R,3S,5S)-3-(3,4-dichlorophenyl)-2-(ethoxymethyl)-8-methyl-8-azabicyclo[3.2.1]octane]) as test compound.

### Protocols

### Testing Tesofensine in Drug Abuse Models

### 1. Self-Administration of Drugs of Abuse in Rats or Primates

### Nicotine Self-Administration Procedure

The intravenous self-administration procedure is based on that described by Shoaib M, Sidhpura N & Shafait S (2003) Investigating the actions of bupropion on dependence-related measures of nicotine in rats. Psychopharmacology 165: 405-412.

Briefly, under surgical anaesthesia rats will be implanted with a chronic Silastic catheter into the external jugular vein. The catheter will be connected to an L-shaped connector (Plastics-One, Roanoke, Va.) that will be mounted in dental cement located on the head of the animal. Daily flushing with 0.9% physiological saline will maintain the patency of the intravenous catheter. Once animals regain body weights above pre-operative weights, the self-administration sessions will start.

Without any food-shaping, in 1-hr limited access sessions, rats will be given the opportunity to lever-press for intravenous infusions of nicotine (0.03 mg/kg/infusion). Once rats show response accuracy with at least 80% of the responses on the active lever and with stable intake of nicotine (±2 infusions) over 2 days, the number of responses required to produce an infusion will be increased progressively up to three (3-response fixed ratio FR-3). Since only 70% of rats normally acquire nicotine self-administration to stable levels (Shoaib M, Swanner L S, Schindler C W, Goldberg S R (1997) Genetic and environmental factors in nicotine self-administration. Addiction 92: 631), for each experiment a group size of 18 rats will be trained thus allowing at least 12 rats to be tested with each compound.

Tests on maintenance (nicotine-taking behaviour) will be conducted in rats that have learned to self-administer nicotine reliably whereby performance is stable under a fixed-ratio 3 schedule of reinforcement (individual session responses within ±20% of the mean for a three-day period). Each dose of the compound will be tested over 3 successive sessions, since extinction takes at least three sessions to reach baseline response levels; there will be at least three days of standard training for baseline behaviour to be regained. A vehicle extinction test may also be conducted to control for between group differences. The order of test doses will be randomised within the treatment group.

### Nicotine Reinstatement Procedure

Rats that have learned to self-administer nicotine reliably whereby performance is stable under a fixed-ratio 3 schedule of reinforcement (individual session responses within ±20% of the mean for a three-day period), the behaviour will then be extinguished. It is important to note that rats self-administering nicotine are not physically dependent to nicotine.

Extinction for all rats will be conducted by not presenting nicotine but also by eliminating the cues that predict the onset of the nicotine injection (infusion pumps are turned off and the stimulus light is left off for the whole session (time-out period not used)). Under these conditions, rates of extinction are much faster and lever-press responses drop to below 20% of nicotine intake baseline levels. Behaviour will be extinguished for at least 6 sessions until criteria are satisfied (individual session responses equal or less than 20% of stable baseline responses, variability within ±20% for a three-day period).

Tests for reinstatement will be conducted by administering nicotine (0.2 mg/kg SC) and presenting cues (stimulus light signalling time-out) and saline infusions (pump noise) contingently upon each lever press response.

The test compound (tesofensine) will be evaluated in a single group of 12 rats to examine cue- and nicotine-induced priming effects. Three doses, plus vehicle of the test compound and a single dose of a positive control compound will be evaluated. Each compound will be administered acutely before priming with nicotine (0.2 mg/kg SC) or saline in the presence of the cues. Five reinstatement tests will be conducted in a randomised sequence for each group: in the proposed model, repeated reinstatement tests are stable which permit a within-subject design to repeatedly test for up to 5 times in a randomised order. If however it is found that the reinstatement effect becomes non-significant following multiple tests, prior to the completion of the five tests, rats will be retrained on nicotine and the re-extinguished. To ensure stability over the course of the experiment, at least 6 days of extinction testing will be allowed for criteria to be met between reinstatement tests.

### Tests on Food-Maintained Responding

In standard operant chambers, food restricted rats will be shaped to lever-press for food in 1-hr limited access sessions. Rats will be given the opportunity to lever-press for food pellets (45 mg). Once rats show response accuracy with at least 80% of the responses on the active lever and with stable number of food pellet presentations over 2 days, the number of responses required to produce a food reward will be increased progressively up to three (3-response fixed ratio FR-3). To equate the number of reinforcers, an extended time-out (maximum of 240 sec) will be utilised. Since behaviour is more stable with food reinforcement, fewer rats will be utilised. Tests with the same doses of tesofensine will be conducted in the same group of rats with each dose tested repeatedly over 3 successive sessions. The order of the doses will be randomised within the group.

Variants of the above self-administration protocol (e.g., a progressive ratio—PR, rather than a fixed ratio—FR schedule) might be used for nicotine or other drugs of abuse (e.g., cocaine, morphine, amphetamine, marijuana, heroin, morphine, alcohol/ethanol) as described by Shaham Y. et al (2003) The reinstatement model of drug relapse: history, methodology and major findings. Psychopharmacology 168: 3-20 and Rollema H. et al (2007) Pharmacological profile of the alpha4beta2 nicotinic acetylcholine receptor partial agonist varenicline, an effective smoking cessation aid. 52: 985-994.

### 2. Conditioned Place Preference

In this model rats or mice are administered a drug of abuse (cocaine, morphine, amphetamine, marijuana, heroin, morphine, alcohol/ethanol) and placed in a distinctive environment, such that after a number of drug-environment pairings rodents will approach and spend more time in the drug associated environment in the absence of the drug. Once this is established the tendency of tesofensine to reduce the propensity of the rodent to approach and spend time in the drug-associated environment will be used as an index of its anti-abuse potential. In addition the ability of tesofensine to reduce acquisition of the drug-environment pairing will be taken as evidence of its ability to reduce learning or re-learning to self-administer drugs of abuse. Additional variants on these procedures will be used as necessary (Le Foll, B. et al. (2005) Dopamine D3 ligands block nicotine-induced conditioned place preferences through a mechanism that does not involve discriminative-stimulus or anti-depressant like effects. Neuropsychopharmacology 30: 720-730).

### 3. Locomotor Sensitisation

In rodents where locomotor sensitisation has been established after chronic administration of nicotine, cocaine, amphetamine or other drugs of abuse, the ability of tesofensine to reduce the sensitised motor response without affecting motility in chronic vehicle treated animals or in normal animals, will be taken as evidence of tesofensine's tendency to reduce incentive-motivational aspects of drug abuse (Tessari et al. M. (2005). Antagonism at metabotropic glutamate 5 receptors inhibits nicotine- and cocaine-taking behaviours and prevents nicotine-triggered relapse to nicotine-seeking. European J. Pharmacology, 499(1-2):121-33).

### 4. Drug Discrimination

In subjects trained to recognise an interoceptive drug abuse cue (cocaine, morphine, amphetamine, marijuana, heroin, morphine, alcohol/ethanol) by pressing on the appropriate lever in a 2-lever operant chamber, tesofensine would be assessed for its ability to reduce preferential responding on the drug-abuse appropriate lever without affecting lever response rate per se (Stolerman I P, D'Mello G D (1981) Role of training conditions in discrimination of central nervous system stimulants by rats. Psychopharmacology 73: 295-303).

### 5. Rodent Models of Drug Withdrawal

Withdrawal after chronic treatment with drugs of abuse (e.g., nicotine, morphine etc) to rodents can lead to a withdrawal syndrome manifest by distinct somatic signs. Withdrawal can also be induced pharmacologically—e.g., mecamylamine administered with rats chronically treated with nicotine. These withdrawal signs can be reduced in their intensity by drugs which are also effective in man—for example, buroprion can attenuate signs of the nicotine withdrawal syndrome in rats (Cryan et al., J. F. (2003) Bupropion enhances brain reward function and reverses the affective and somatic aspects of nicotine withdrawal in the rat). Tesofensine's tendency to reduce nicotine or other drugs of abuse (e.g., opiate) induced withdrawal signs can be assessed in rodents using available rating scales or using affective models (Malin, D. H. (2001) Nicotine dependence: studies with a laboratory model. Pharmacology, Biochemistry and Behaviour. 70: 551-559; Cryan et al., supra).

### BRIEF DESCRIPTION OF THE DRAWING

The present invention is further illustrated by reference to the accompanying drawing, in which

FIGS. 1 and 2 show the effects of Tesofensine on nicotine self-administration. B1, B2 and B3 are the baseline sessions; T1, T2 and T3 are the test sessions; and R1 and R2 are the recovery sessions. The amount of nicotine administered is 0.03 mg/kg animal/infusion unit. FIG. 1 shows the result from 1 mg/kg SC and FIG. 2 shows the result from 3 mg/kg SC.

### Test Data

### Testing Tesofensine in the Nicotine Self-Administration Procedure in Rats

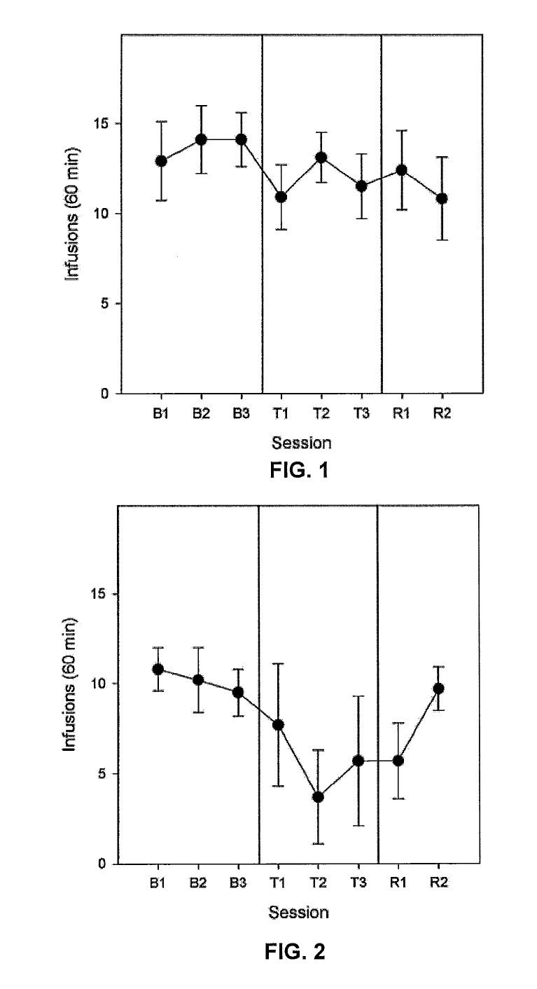

Tesofensine was tested according to the protocol as described above. Data from tests with Tesofensine (1 mg/kg SC and 3 mg/kg SC) tested in 7-8 rats per dose are presented in FIGS. 1 and 2. The amount of nicotine administered (in 60 minutes) is 0.03 mg/kg animal/infusion unit.

1. Method for treating addiction comprising administering to a human a composition comprising a compound of formula I 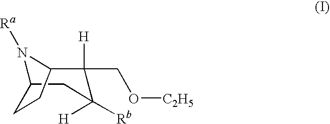
wherein
R^{a} represents hydrogen or alkyl;
R^{b} represents a dihalophenyl group;
any of its stereoisomers or any mixture of its stereoisomers, or a pharmaceutically acceptable salt thereof in a therapeutically-effective amount in the range of about 0.1-2 mg API daily.

2. The method of claim 1 wherein R^{a} represents hydrogen or methyl.

3. The method of claim 1, wherein R^{b} represents 3,4-dichlorophenyl.

4. The method of claim 1 wherein the compound of formula I is
tesofensine [(1R,2R,3S,5S)-3-(3,4-dichlorophenyl)-2-(ethoxymethyl)-8-methyl-8-azabicyclo[3.2.1]octane]; or
(1R,2R,3S,5S)-3-(3,4-dichlorophenyl)-2-(ethoxymethyl)-8-azabicyclo[3.2.1]octane;
or a pharmaceutically acceptable salt thereof.

5. The method according to claim 1, wherein the daily dosage of the compound of formula I is about 0.25-1.0 mg API daily.

6. The method according to claim 1, wherein the addiction is selected from the group comprising nicotine addiction, alcohol addiction, drug addiction, such as opioid addiction, nicotine craving, alcohol craving, and drug craving, such as opioid craving, food craving, craving for fatty food and craving for carbohydrate rich food.

7. The method according to claim 1, wherein the composition is administered orally, intravenously, intravascularly, intraperitoneally, sub-cutaneously, intramuscularly, inhalatively, topically, by patch, or by suppository.

8. A pharmaceutical composition for the treatment of addiction in a human, said composition comprising a therapeutically-effective amount in the range of about 0.1-2 mg API daily of a compound of formula I 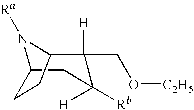
wherein
R^{a} represents hydrogen or alkyl;
R^{b} represents a dihalophenyl group;
any of its stereoisomers or any mixture of its stereoisomers, or a pharmaceutically acceptable salt thereof in admixture with one or more pharmaceuticaly acceptable adjuvants, excipients, carriers and/or diluents.

9. The composition according to claim 8, wherein R^{a} represents hydrogen or methyl.

10. The composition according to claim 8, wherein R^{b} represents 3,4-dichlorophenyl.

11. The composition according to claim 8, wherein the compound of formula I is
tesofensine [(1R,2R,3S,5S)-3-(3,4-dichlorophenyl)-2-(ethoxymethyl)-8-methyl-8-azabicyclo[3.2.1]octane]; or
(1R,2R,3S,5S)-3-(3,4-dichlorophenyl)-2-(ethoxymethyl)-8-azabicyclo [3.2.1]octane;
or a pharmaceutically acceptable salt thereof.

12. The composition according to claim 8, wherein the daily dosage of the compound of formula I is 0.25-1 mg API daily.

13. The composition according to claim 8, wherein the addiction is selected from the group comprising nicotine addiction, alcohol addiction and drug addiction, such as opioid addiction

14. The composition according to claim 8, wherein the composition is administered orally, intravenously, intravascularly, intraperitoneally, sub-cutaneously, intramuscularly, inhalatively, topically, by patch, or by suppository.

15. (canceled)
